(19) United States
(12) Patent Application Publication
(10) Pub. No.: US 2013/0218297 A1
(43) Pub. Date: Aug. 22, 2013

(54) PROSTHETIC FOOT SHELL ENABLING RAPID CONVERSION BETWEEN SHOE AND BAREFOOT WALKING

(71) Applicants: Elwin Isaac Nordman, JR., San Jose, CA (US); Gary M. Berke, San Carlos, CA (US)

(72) Inventors: Elwin Isaac Nordman, JR., San Jose, CA (US); Gary M. Berke, San Carlos, CA (US)

(21) Appl. No.: 13/854,680

(22) Filed: Apr. 1, 2013

Related U.S. Application Data
(63) Continuation-in-part of application No. 13/365,046, filed on Feb. 2, 2012.

(51) Int. Cl. A61F 2/66

(52) U.S. Cl. CPC A61F 2/66
USPC 623/53

(57) ABSTRACT

An easily removable/rapidly replaceable foot shell device for a prosthetic leg, and method for using the device. In a preferred embodiment, a plurality of foot shell devices, often with at least differing heel heights adjusted for barefoot walking, or for use with shoes with different heel heights, will be provided. The devices are designed to allow an average adult to remove a foot shell and replace a foot shell within a short span of time, such as under two minutes. Such a plurality of devices can help a prosthetic leg user avoid prosthetic leg mal-adjustment problems such as hyperextended knee pressure or prosthetic posterior lean. The devices can also adjust the height of the prosthetic foot to compensate for the missing elevation caused by a missing shoe when walking barefoot. The device may be further configured with one or more fasteners to facilitate rapid application and removal.

### CROSS REFERENCE TO RELATED APPLICATIONS

This application is a continuation in part of application U.S. Ser. No. 13/365,046, “PROSTHETIC FOOT COVERING ENABLING RAPID CONVERSION BETWEEN SHOE AND BAREFOOT WALKING”, inventor Elwin Isaac Nordman, Jr., filed Feb. 2, 2012; the contents of this application are incorporated herein by reference.

### BACKGROUND OF THE INVENTION

1. Field of the Invention

This invention is in the field of prosthetics and prosthetic foot shells for lower limb amputee patients.

2. Description of the Related Art

Human walking is a complex cyclical series of body movements in which the weight of the body is sequentially supported by first one leg and then the other. As the weight of the body is supported by one leg during the stance phase portion of the walking or gait cycle, the other leg swings forward so that it in turn can be later used for weight support during a later part of cycle. During this gait cycle, the pelvis rotates in various planes. At the knee joint, during normal walking, the knee flexes slightly at initial contact then extends to near neutral (180°) during the remainder of the stance phase.

The angle of the ankle and foot also change during the gait cycle. At the beginning of the stance phase, the foot flexes about the ankle so that the heel of the foot strikes the floor first, followed by rapid motion of the foot in plantar-flexion to be flat the floor. During the later part of the stance phase, the foot then rotates further relative to the ankle, so that the toe of the foot remains in contact with the floor while the heel elevates. These various bone and joint movements are controlled by various muscles that typically operate both above and below their respective joints. These muscles in turn are under precise control by nerves and the body's various natural sensors. Amputation literally cuts through this delicate biomechanical mechanism with a knife, and throws the entire process out of kilter.

There are various types of lower limb amputations. In some cases, only one limb is amputated (unilateral amputation), while in other cases both limbs are amputated (bilateral amputation). In this discussion, lower limb amputation will be assumed to mean that at least the natural foot and ankle has been removed. Irrespective of the cause of limb loss, whenever possible, surgeons will attempt to preserve the patient's natural knee joint, and lower limb amputees who retain their natural knee joint and at least some of the natural tibia bone are called below-knee amputees or “transtibial” amputees. By contrast, when the knee and portions of the femur must be amputated, these amputees are termed “transfemoral” or above knee (mid thigh) amputees. In either case, either during the initial amputation surgery, or in later subsequent surgeries, the remaining bone, muscle and skin at the severed end of the limb are configured into a stump that in turn can be fit into the weight being socket of an appropriate lower-limb prosthetic device. There are other methods of amputation (e.g. higher and lower) as well, and indeed some individuals are born with a missing portion of a leg or foot, however since amputation is the most common mechanism here, it is simpler to speak of all of these different causes together as “lower limb amputation”, rather than to discuss each case individually.

As might be imagined, lower limb amputation greatly interferes with the complex series of events that occur during human walking, and much of the art and science of lower limb prosthetic design has been focused on providing artificial mechanisms that, working in concert with the amputee's remaining natural muscles and tissues, can provide as adequate a substitution for a natural and comfortable gait as possible.

Although the technology of lower limb prosthetics design has advanced to the point where, in many cases, the performance of the prosthetic limb can be remarkably natural, the limitations of even modern prosthetics still must be appreciated. The lower limb amputee has to walk without the aid of many important muscle groups, and without the aid of his or her natural lower extremity nerves and natural kinesthetic sensors (e.g. sense of touch, kinesthetic senses, and the like). The amputee's lower limb stump or stumps have to bear weight in a biologically unnatural manner, and can be damaged if not treated carefully. As a result, prosthetic specialists must precisely design, adjust and align the amputee's lower limb prosthetics to various parameters, including the height characteristics of the shoes that the prosthetic patient intends to wear. This is true regardless of level of amputation, from ankle disarticulation through hip disarticulation.

Mal-adjusted or misconfigured lower limb prosthetics can produce a number of problems. One problem, for example, is the problem of knee joint hyperextension pressure (or more accurately, pressure on the knee to move in a hyperextension direction). In a knee joint hyperextension situation, particularly during the initial portions of the stance phase, the angle of the knee may undergo significant pressures on the front portion, attempting to push the knee to extend beyond 180°, thus imposing a very unnatural stretching force on the knee. This problem is particularly acute for transtibial amputees who have retained their natural knee joint or joints, because knee hyperextension can damage precious natural tissue. However even for trans-femoral amputees, knee hyperextension moments are undesirable as well as they create difficulty in walking and pain in the hip region.

Such knee joint hyperextension pressures often occur when, for example, an amputee attempts to walk barefoot on a lower limb prosthetic that has been adjusted for walking with shoes with a certain shoe heel height. It can also happen when an amputee attempts to transition from one set of shoes with a certain heel and sole height to a second set of shoes that may have at least a different sole height.

Another problem that can occur with lower limb amputees when walking shoeless with a lower limb prosthetic otherwise configured for walking with shoes is the problem of prosthetic posterior lean. Here the lower limb prosthetic, normally adjusted for walking with shoes with a certain shoe heel height, tends to fall or lean backwards while standing barefoot. This again can cause damage to the amputee's remaining natural tissue, make walking difficult and painful as well as increasing the risk of a fall.

As a result of these and other considerations, lower limb amputees are typically given prostheses adjusted for wear with particular type of shoes with a particular shoe height and heel height. Lower limb amputees are further instructed by their prosthetics clinicians to always wear shoes with the same approximate shoe and shoe heel height, and to not attempt to either walk barefoot (i.e. without shoes), or to attempt to walk in shoes with a substantially different sole height and shoe heel height.

Enhancing the Cosmetic Appearance of Lower Limb Prosthetics:

Changing for the moment to a different type of prosthetic foot problem, lower limb prostheses are often designed from a functional mechanical engineering basis, and often their unadorned cosmetic appearance looks mechanical and very unnatural. An additional problem is that the mechanical portions of a prosthetic foot may not always be directly compatible with standard shoes, which the amputee will often wish to wear to preserve a natural looking appearance.

To help improve this functional and cosmetic appearance, the artificial foot of a lower limb prosthetic will often be sold with (or at least provided on an aftermarket with) the mechanical portions of the artificial foot covered by a foot shell. A foot shell is a hollow, relatively thick, skin-colored plastic covering, usually semi-rigid but not completely rigid, usually with an exterior that has been molded to resemble the general shape of a natural foot (i.e. often with toes). The foot shell is designed to allow the artificial foot to fit into the shoe or shoes that the lower limb amputee intends to wear. Often this foot shell will often terminate at about the ankle level of the prosthetic foot, exposing various mechanical shafts, bolts, and other fixtures above the shell to the outside world. This is not as big of an issue, since the wearer will often be wearing clothing, such as pants, that cover this mechanical portion, and/or will have a cosmetic portion (e.g. foot covering and/or shin covering) added to cover the exposed components.

Other prosthetic foot coverings: As previously discussed in parent application U.S. Ser. No. 13/365,046, the contents of which are incorporated herein by reference, and elsewhere, it is relatively common to further cover the prosthetic foot and foot shell with a thin, flexible, “skin” or covering. With the exception of the coverings disclosed by U.S. Ser. No. 13/365,046 however, such coverings, discussed in more detail in U.S. Pat. Nos. 3,400,408; 5,133,775; 5,593,453, 6,153,139; and 6,911,049 were also used to improve the cosmetic appearance of prosthetic feet, but were typically too thin to contribute towards any meaningful correction of the user's gait.

Prior art foot shells were designed for secure attachment to the mechanical portions of the artificial foot, and it took a considerable amount of effort to attach or remove a foot shell from the mechanical portions of the artificial foot. Indeed, as a rule, generally only prosthetic specialists would normally replace prior art foot shells. This was because these prior art foot shells were designed in a manner that often required both specialized tools, and a considerable amount of strength, (and risk of injury) to remove the foot shell from a prosthetic foot. Replacing either that foot shell or another foot shell back on the prosthetic foot also required considerable expertise and often tools and strength.

As a result, after the patient had become accustomed to his or her artificial foot, often the prior art foot shells were examined or replaced only at intervals of many months such as every six months. Prior art foot shells often had use a lifetime or around six to 12 months, considerably less than that of the underlying prosthetic foot. Thus on a typical six month's practitioner visit, the practitioner would either remove the old foot shell and replace with a fresh foot shell, or alternatively (if the foot shell was in good shape) replace the old foot shell again.

To reduce noise and wear between the prosthetic foot and the foot shell, it is also common in the art to put a sock, such as a fabric sock, over the prosthetic foot before the foot is applied to the foot shell. One problem with prior art foot shells, however, is that due to the long periods of time between foot shell replacement, these socks were in turn worn on the artificial foot for many months, often developing a distressing foul odor as a result.

### BRIEF SUMMARY OF THE INVENTION

In one embodiment, the invention may be an easily removable/rapidly replaceable foot shell device for a prosthetic leg, and method for using the device. In a preferred embodiment, a plurality of foot shell devices, often with at least differing heel heights adjusted for barefoot walking, or for use with shoes with different heel heights, will be provided. The devices are designed to allow an average adult to remove a foot shell and replace a foot shell within a short span of time, such as two minutes. Such a plurality of devices can help a prosthetic leg user avoid prosthetic leg maladjustment problems such as hyperextended knee or prosthetic posterior lean. The devices can also adjust the height of the prosthetic foot to compensate for the missing elevation caused by a missing shoe when walking barefoot. The device may be further configured with one or more fasteners to facilitate rapid application and removal.

The invention is based, in part, on the insight that although from a functional standpoint, it is often inadvisable for a lower limb amputee to walk “barefoot”, that is to attempt to walk without shoes on a lower limb prosthetic adjusted for shoes, socially this is sometimes hard to avoid.

For example, in some households, it is considered inappropriate to wear shoes indoors. A lower limb amputee confronted with such a situation is presently at a loss as to what to do. It is difficult to knowingly enter into a situation where the risk of damage and falls is significant. Even if the lower limb amputee were to assume the risk of complications, such as knee hyperextension and prosthetic posterior lean, without the shoe, the mechanical nature of the prosthetic limb becomes more clearly revealed, leading to more social awkwardness.

In other situations, such as poolside parties, athletic clubs, and the like, prosthetic users are under social pressure both to walk barefoot, and also to expose more skin than normal. Here again, these situations expose the lower limb amputee to both risk of damage due to knee hyperextension and prosthetic posterior lean, as well as and more social awkwardness due to more prominent exposure of the mechanical nature of the prosthetic.

Further, in almost all situations, a foot shell with a moldy, wet, dirty or mal-odorous internal sock is also undesirable. The invention is based, in part, on the insight that a rapidly replaceable (i.e. remove and attach again) foot shell would allow prosthetic foot socks to be replaced on a more frequent schedule, thus diminishing this undesirable problem.

The invention is based, in part, on the insight that what is needed is a new or improved type of rapidly replaceable foot shell that, either alone, or often as part of a kit with various foot shells with different heights, can be rapidly interchanged on a lower limb prosthesis.

The invention is based, in part, on the insight that it would be desirable to provide a plurality of foot shells (e.g. a kit of at least two foot shells with at least differing heel heights) that are designed for rapid and easy attachment and detachment from the underlying mechanisms of the artificial foot. Such easy and rapid attachment and removal can be performed by various techniques, such as providing one or more slits and optional fasteners in the foot shell to make the foot easier to deform while taking on and off.

Thus the invention may comprise a foot shell that is configured to cover the mechanical portions of at least the prosthetic foot. In this case, one version of the foot shell might, for example, be configured for walking with shoes, while an alternate version of the foot shell would have a higher or thicker internal heel or sole such that when the foot shell is applied to the mechanical portions of the prosthetic foot, the foot shell elevates at least the heel (or both heel and sole) of the prosthetic foot to a sufficient height above the floor as to reduce the risk of knee hyperextension or prosthetic posterior lean when the amputee walks barefoot.

Alternatively, the invention may provide several types of rapid replacement foot shells, where one type may be configured for walking barefoot (e.g. it could have a very thick heel and possibly thick sole as well, so as to elevate at least the heel of the prosthetic foot to a sufficient height above the floor as to reduce the risk of knee hyperextension or prosthetic posterior lean when the amputee walks barefoot), and other types could be configured for various shoes with multiple heel heights. (i.e. moving from a dress shoe to a tennis shoe to barefoot)

Thus the invention's prosthetic foot shell device would, on the one hand, be designed to compensate for the differences in heel height and other parameters caused by the missing shoe, while on the other hand also be designed to closely resemble the outside of a normal foot (e.g. have an exterior color that resembles the user's normal skin color). This would enable a lower limb amputee to both avoid risk of damage while walking barefoot (e.g. without shoes), and also help enable the lower limb amputee provide a more cosmetically natural appearance under shoeless situations as well.

Thus in one embodiment, the invention may be device and method for providing and using one or more rapidly or easily removable foot shell devices for a lower limb prosthesis, such as a prosthetic leg or foot (here the terms lower limb prosthesis and prosthetic foot will often be used interchangeably).

The device will enable the same prosthetic foot, without readjustment, to be used by an amputee with a prosthetic foot for either walking with shoes having shoe heels and other shoe elevation, or walking barefoot with minimal risk of knee hyperextension or prosthetic posterior lean.

### BRIEF DESCRIPTION OF THE DRAWINGS

FIG. 1 shows the positions of the major parts of the legs during the stance and swing phases of the normal gait cycle. Note that even during the stance phase, the angle of the knee is normally less than 180°, thus allowing the knee of the stance (weight bearing) leg to bend forward slightly and absorb some of the shock of supporting weight.

FIG. 2 shows a common gait abnormality often caused by a maladjusted prosthetic leg, such as can happen when an amputee using a prosthetic leg adjusted for walking with shoes attempts to walk without the shoes. Here during stance phase, the angle of the knee becomes hyperextended (e.g. the pressure pushes the knee to hyperextension) to or beyond 180°. This can result in prosthetic posterior lean, and additionally (particularly if the walker retains a natural knee joint) puts unwanted stress on the amputee's already overstressed natural knee and hip tissues.

FIG. 3 shows one mechanism by which the invention may act to reduce the problem of knee hyperextension. In this embodiment, various easily removable foot shells with varying height (often varying heel height) are placed on the prosthetic foot, and for example adjust the elevation of the prosthetic foot to compensate for the missing shoe. These foot shells may optionally also providing a cosmetic cover that better resembles the appearance of the user's natural foot skin.

FIG. 4 shows the interior and exterior of a foot shell while being worn on a prosthetic foot, as well as the effect of a higher foot shell heel height on the angle of the prosthetic foot's shank.

FIG. 5 shows a rapidly replaceable foot shell with a heel side slit, and a butterfly type latch or clip disposed on opposite sides of the heel side slit. The latch or clip is shown in both an open and closed configuration, and one of the sides of the heel slit is shown being deformed under the force of normal human hand pressure. The prosthetic foot is here shown covered with a sock.

FIG. 6 shows a rapidly replaceable foot shell with a forefoot side slit, and a butterfly type latch or clip disposed on opposite sides of the forefoot side slit. This latch or clip is also shown in both an open and closed configuration and one of the sides of the forefoot slit is shown being deformed under the force of normal human hand pressure. The prosthetic foot is here shown covered with a sock.

FIG. 7 shows a prosthetic foot and a kit of three different foot shells, each with a somewhat different heel height, ranging from a low heel height, a medium heel height, and a higher heel height. The sole of the foot shell may also be adjusted in height as desired. Note that even the higher heel foot shell models may optionally retain a generally normal foot cosmetic appearance.

### DETAILED DESCRIPTION OF THE INVENTION

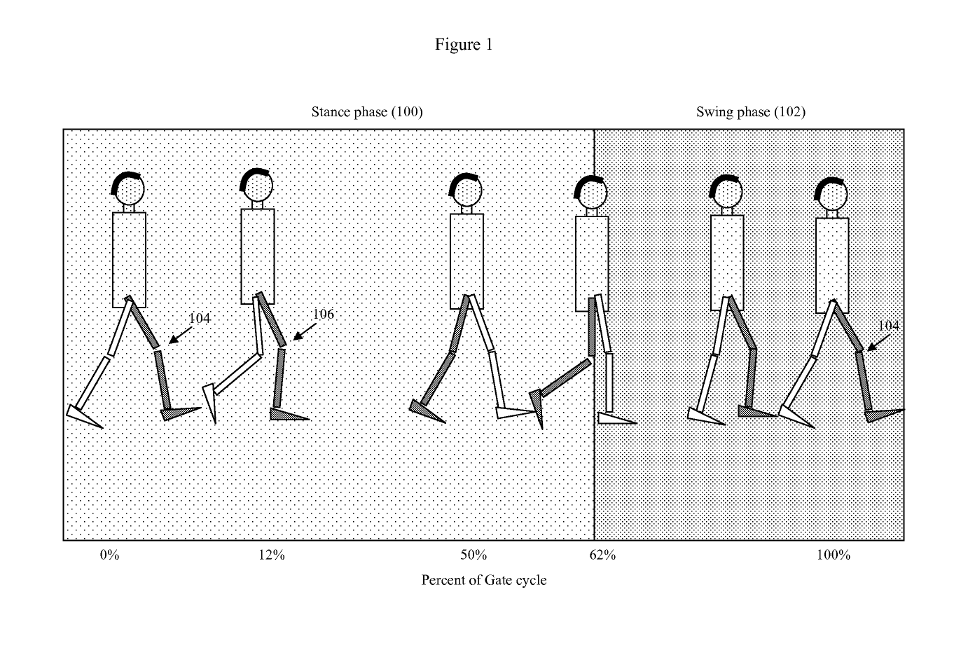

In order to better show the purpose and mode of operation of the invention, a simplified diagram of an ideal human gait cycle is shown in FIG. 1, which shows the positions of the major parts of the legs (e.g. the femur, the tibia, the knee, ankle and foot) during the stance (100) and swing (102) phases of the normal gait cycle. Note that in a proper or normal stance phase, the angle of the knee (104), (106) will be somewhat less than 180°, thus allowing the knee of the stance (weight bearing) leg to bend forward slightly, and absorb some of the shock of supporting the weight of the walkers body.

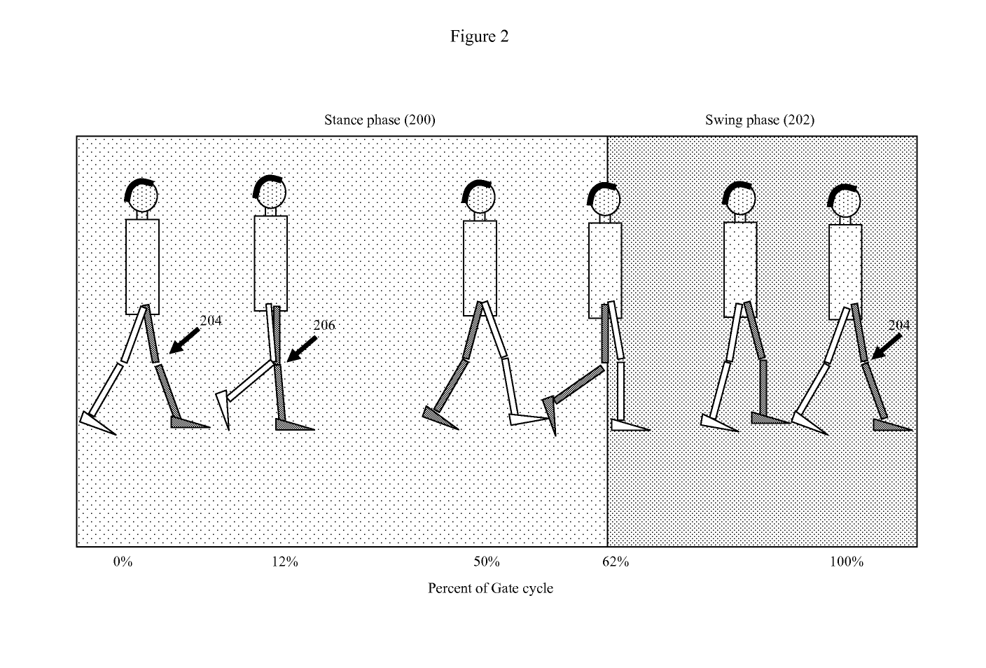

By contrast, FIG. 2 shows a common gait abnormality that is often caused by a misadjusted prosthetic leg. Here again, both stance phase (200) and swing phase (202) are shown. This problem frequently happens when an amputee using a prosthetic leg that is otherwise properly adjusted for walking with shoes attempts to walk without the shoes. Here during the stance phase (200), the angle of the knee succumbs to hyperextension moments forcing the knee to or beyond 180° (204), (206). This results in several problems, including prosthetic posterior lean. Additionally, if the amputee is a transtibial amputee with a functional natural knee joint, this knee hyperextension can put unnatural pressure on the amputee's natural knee tissue, resulting in higher risk for subsequent knee joint damage and pain.

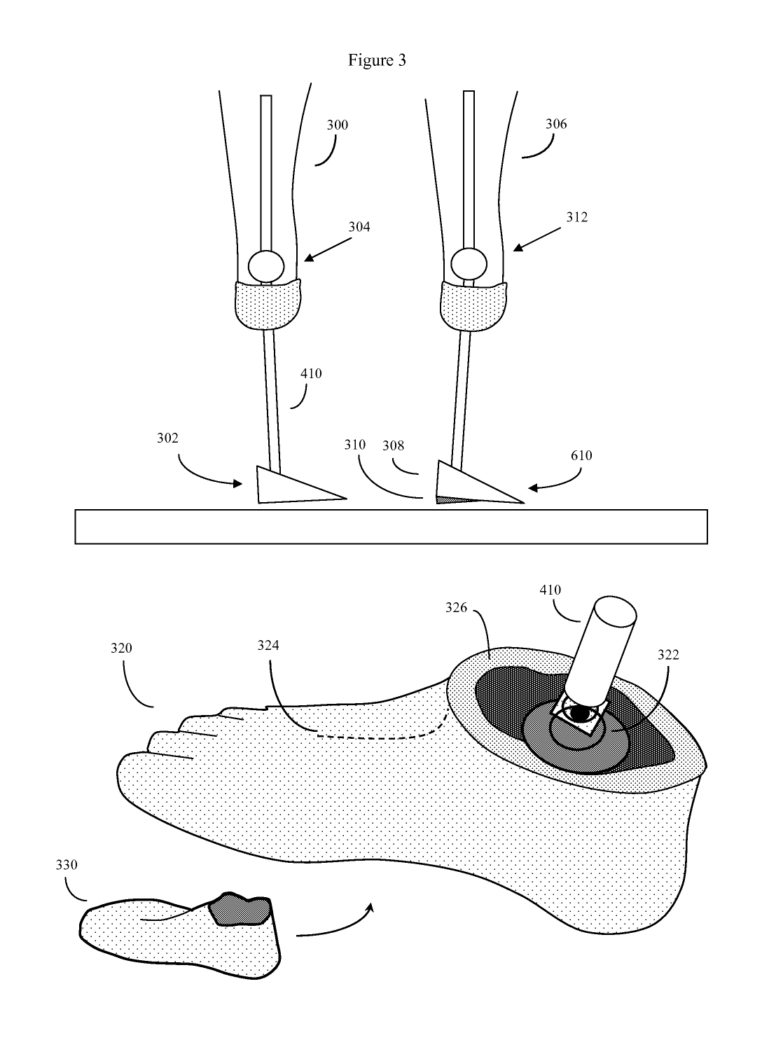

As previously discussed, the mechanical portions of the artificial foot are usually covered by a foot shell, here shown in FIG. 3 (320) which is a relatively thick plastic covering, often skin colored, that is molded in the general shape of a natural foot (i.e. often with toes). The foot shell is designed to allow the artificial foot to fit into the shoe that the lower limb amputee intends to wear (not shown). At present, as previously discussed, foot shells are generally designed for very secure attachment to the mechanical portions of the artificial foot (322), and it takes a considerable amount of effort to attach or remove a foot shell (320) from the mechanical portions of the artificial foot.

As previously taught in parent application 13/365,046, however, a foot shell may be redesigned for easier attachment and detachment from the underlying mechanisms of the artificial foot. One way to do this is by forming a slit (324) in the foot shell to make the sides of the foot shell easier to deform while taking on and off. Such rapidly removable foot shells thus allow the user to have more than one foot shell on hand for any given prosthetic foot. One foot shell, for example, may be configured for barefoot walking (e.g. 308, 310), while others (e.g. 302) may be configured for walking in shoes with varying heel heights and sole heights.

More specifically, FIG. 3 shows one mechanism by which the invention may act to reduce the problem of knee hyperextension pressure. In FIG. 3 (300), a transtibial amputee with a functional natural knee joint is attempting to walk barefoot using a prosthetic leg and a prior art non-rapidly replaceable foot shell adjusted for walking with shoes (302). As a result, some knee hyperextension (304) occurs during stance phase.

By contrast, in (306), the same amputee is walking with same prosthetic foot, that has been rapidly converted to walking in barefoot mode using the invention's replaceable foot shell. This replaceable foot shell (308) that now has at least an alternate heel height (310) and often also an alternate sole height as well. Due to this alternate heel and/or sole height, during stance phase, the foot shell's optimized heel and sole height helps position the amputee's foot at a more favorable angle, thus reducing the problem of knee hyperextension and potential leg length discrepancies. (612).

Note that for the purposes of this disclosure, it is generally irrelevant if the prosthetic foot user is further covering their prosthetic foot with a prior art thin flexible foot covering (e.g. “foot skin”) or not, because the change in height and gait caused by most of these prior art thin flexible foot coverings was generally small. An example of such a thin flexible foot cover or “foot skin” is shown as FIG. 3 (330) (not to scale). Thus for example, when the present disclosure speaks of “barefoot walking”, or use of shoes with different heel heights or sole heights, it generally does not matter if the user has further covered the foot shell with a thin flexible cover or not. Thus any teaching in this regard herein should be considered to be contemplating both possibilities, both with and without the thin flexible cover or “foot skin”.

However in the case, such as discussed in U.S. Ser. No. 13/365,046, the normally thin prosthetic foot covering may have a significant heel height of its own, and is designed to make a significant contribution to correcting the user's gait, then the teaching of the present foot shell disclosure is still valid, however the corrective effect of the thicker heel flexible foot covers of Ser. No. 13/365,046 and the present foot shells will generally have an additive effect, so that, for example, a larger foot heel height correction can be achieved by any combination of the heel heights of the present replaceable foot shells, plus the heel heights of the thicker heel flexible foot covers disclosed in Ser. No. 13/365,046. Thus although here again, the present foot shell teaching should be considered to be contemplating both possibilities, with and without foot cover or “foot skin” use, the magnitude of the correction of the foot shell heights (e.g. heel heights) may be lesser if use of the invention's foot shells in conjunction with a gait correcting foot skin is also contemplated.

Thus the invention may comprise a rapidly replaceable foot shell that is configured to cover the mechanical portions of at least the lower portion of the prosthetic foot. In some embodiments, one version of the rapidly replaceable foot shell would be configured for walking with shoes, while an alternate version of the rapidly replaceable foot shell would have an internal heel or sole insert with a height optimized for barefoot walking That is, when the barefoot optimized foot shell is applied to the mechanical portions of the prosthetic foot, the foot shell elevates at least the heel of the prosthetic foot to a sufficient height above the floor as to reduce the risk of knee hyperextension or prosthetic posterior lean when the amputee walks barefoot.

Alternatively, an alternate version of the foot shell configured for walking barefoot could have a very thick heel and possibly thick sole as well, so as to elevate at least the heel of the prosthetic foot to a sufficient height above the floor as to reduce the risk of knee hyperextension or prosthetic posterior lean when the amputee walks barefoot.

As previously discussed, that although heel height adjustments will primarily be used in this disclosure as an example of this type of shoe height adjustment, other types of shoe height adjustment, such as overall shoe sole height, may also be compensated for by appropriately configuring the prosthetic foot shell. Thus in this discussion, all shoes are presumed to have at least shoe heels with a shoe heel height of at least ¼″ to ⅜″, but these shoes may also have an overall sole height as well throughout, and the insert may include this overall sole height adjustment as well.

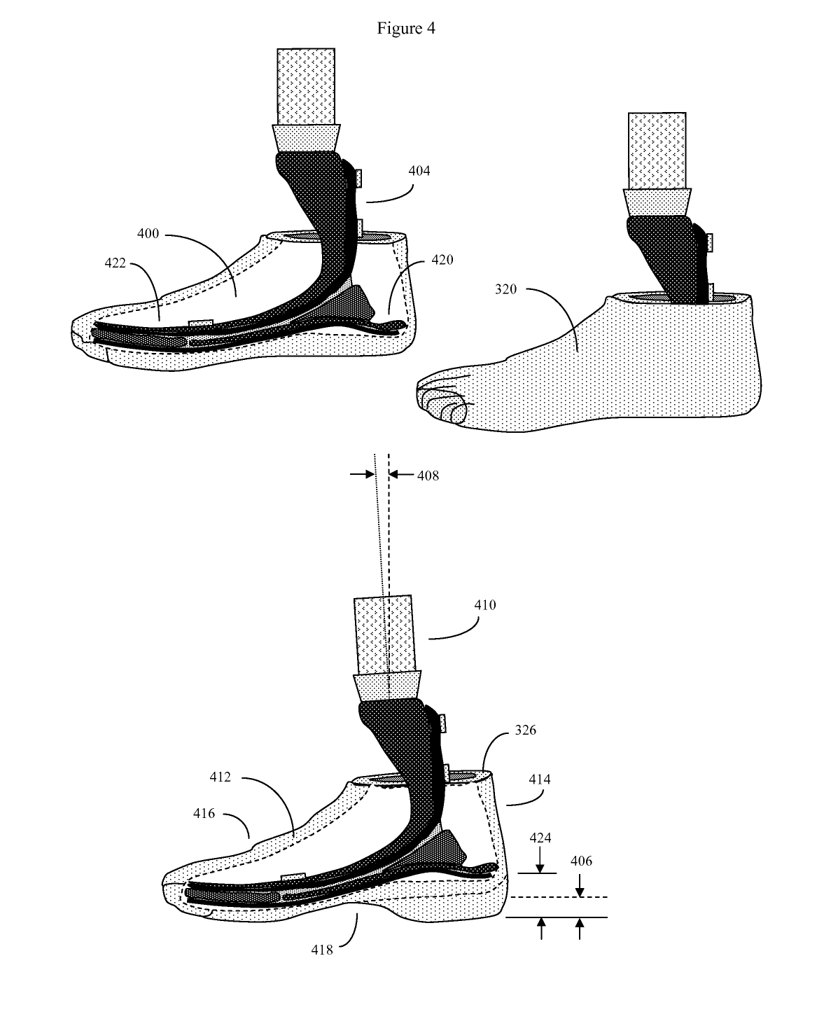

FIG. 4 shows the interior (400) and exterior (320) of a foot shell while being worn on a prosthetic foot (404), as well as the effect of a higher foot shell heel height (406) on the angle (408) of the prosthetic foot's shank (410).

Thus in some embodiments, the invention may be a rapid release hollow (400) foot shell (320) for a mechanical prosthetic foot (404). This foot shell will generally comprise a hollow shell (412) with an exterior approximately in the shape of a human foot. This hollow shell will generally in turn comprise a heel side portion (414), a forefoot side portion (416), and a sole portion (418).

This hollow shell (412) will have a hollow interior (400) configured to substantially surround at least the heel portion (420) and the forefoot portion (422) of a prosthetic foot comprising a heel portion (420), a forefoot portion (422), and a shank (410).

Typically, the hollow shell of the foot shell will have a heel height (424) representing the distance that the foot shell (320) elevates the heel portion (420) of the prosthetic foot (404) when the foot shell (402) is mounted on the prosthetic foot (404).

The hollow shell of the foot shell will further generally have an upper rim (326) disposed in a single closed loop that allows at least the shank portion (410) of the prosthetic foot (404) to extend outside the foot shell (320) when the prosthetic foot is disposed inside of the foot shell (320).

As previously discussed, to enable the foot shell to be rapidly replaced, according to the invention, the hollow shell of the foot shell will additionally have one or more slits extending entirely through the wall of the hollow shell. The idea here is that if the foot shell is made of a semi-rigid plastic, capable (for example) of deforming upon sufficient application of human hand applied force, then the one or more slits, if properly placed and sufficiently long, can be temporarily widened by the application of human hand pressure. In alternative embodiments, a tool may also be used to temporarily widen the one or more slits. However for convenience, often a foot shell design that can be rapidly replaced using normal human hand pressure is preferred.

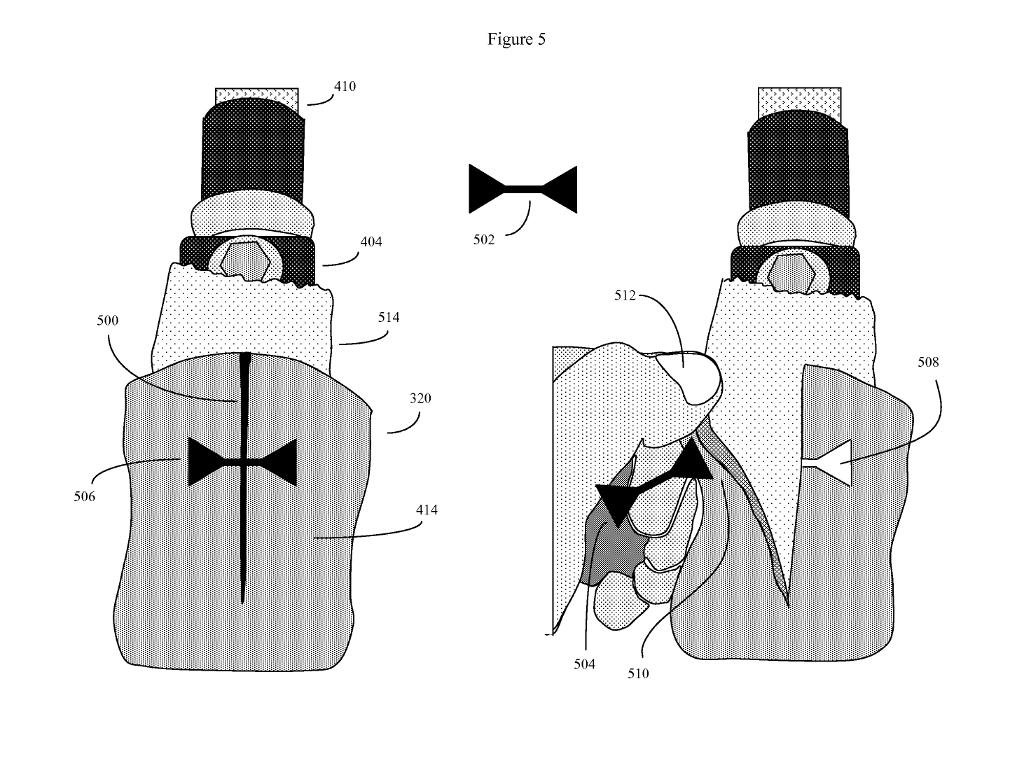

One or more various types and positions of slits may be used. For example, FIG. 5 shows one embodiment, where the invention's rapidly replaceable foot shell (320) with a heel side (414) slit (500), are held together with a butterfly or other design type latch or clip (502) disposed on opposite sides of the heel side slit (500). This latch or clip (502) is shown in both an open (504) and closed (506) configuration. To accommodate this latch or clip, the surface of the foot shell (412) may, in some embodiments, have one or more holes, detents, or recesses (508) in order to accommodate the clip (602) in a manner that preferably will allow the clip to be flush with the surface of the foot shell when the clip is in a closed configuration (506).

On the right side of FIG. 5, one of the sides of the heel slit (510) is shown being deformed under the force of normal human hand pressure (512).

The prosthetic foot is shown here covered with a sock (514).

In some embodiments, more than one slit may be formed on the back side of the foot shell, to further make expansion easier. Alternatively, these one or more slits may be cut in such a way as to form a tongue or slot.

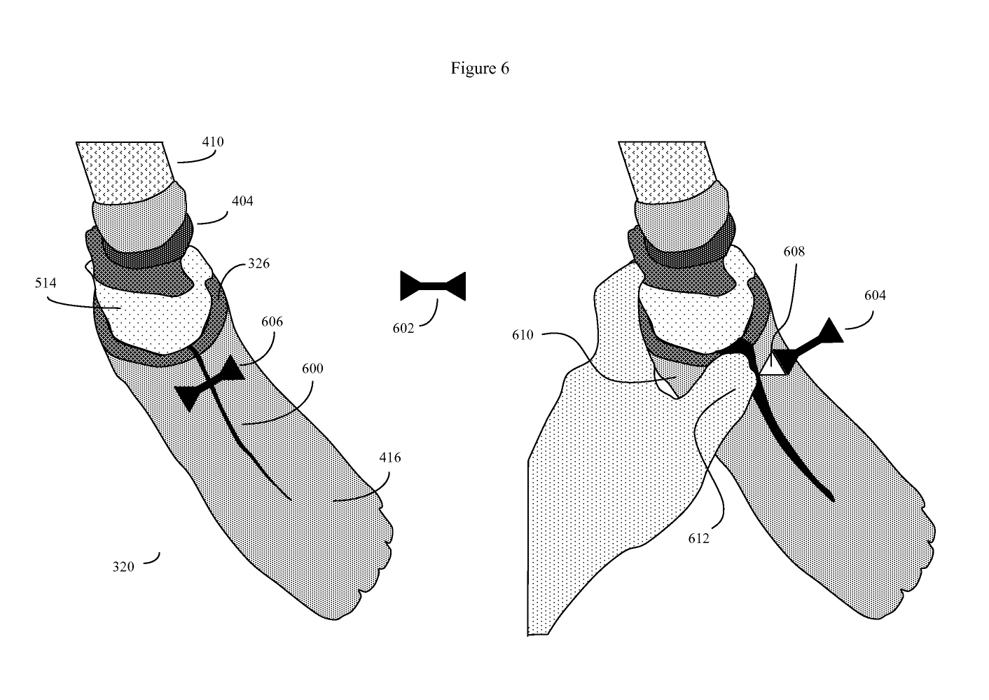

Other slots (600) may alternatively or also be formed on the front (forefoot) side of the foot shell (416), as is shown in FIG. 6.

FIG. 6 shows a rapidly replaceable foot shell (320) with a forefoot side slit (600), and a butterfly type latch or clip (602) disposed on opposite sides of the forefoot side slit (600). This latch or clip is also shown in both an open (604) and closed (606) configuration. As previously discussed, the surface of the hollow foot shell (412) may, in some embodiments, have one or more holes, detents, or recesses (608) in order to accommodate the clip (602) in a manner that preferably will allow the clip to be flush with the surface of the foot shell when the clip is in a closed configuration (606). Other clip devices, such as hinges, may also be used (not shown).

In FIG. 6, one of the sides of the forefoot slit (610) is shown being deformed under the force of normal human hand pressure (612).

The prosthetic foot is here also shown covered with a sock (514).

Thus according to the invention, the improved rapidly replaceable foot shell will generally comprise at last one slit in the foot shell's wall. This slit may be at least one heel side slit (500), usually with a length of at least 1″, and with a length that may further extend 1.5″, 2.0″, 2.5″ or more from the upper rim (326) towards the sole (418) of the foot shell.

As a rule, longer heel slit lengths generally will make the foot shell easier to “open”, however if the slit becomes too long, the stability of the foot shell while mounted on the prosthetic foot may be adversely affected. Thus the optimum slit length may still fall short of covering the complete length of the heel portion of the foot (414).

The at least one slit may also or alternatively be, at least on some embodiments, at least one forefoot side (416) slit (600) of at least 1.5″ length, and often 2″, 2.5″ or longer extending from the upper rim (326) towards the forefoot portion (416) of the foot shell. Here as well, longer forefoot slit lengths generally will make the foot shell easier to “open”, however if the slit becomes too long, the stability of the foot shell while mounted on the prosthetic foot could be adversely affected, thus there may be an optimum slit length that falls short of covering the complete length of the forefoot portion of the foot (416).

Foot shells (e.g. the walls of the foot shell) will often be made of semi-rigid/semi-deformable plastic materials such as various synthetic polymers and copolymers such as polyethylene, polypropylene; and various (and often proprietary) polymer, copolymer, and composite blends. In a preferred embodiment, the hollow plastic foot shell wall material (412), at least in the regions proximate one or more of the slits (e.g. 500, 600) will be formed from a deformable or semi-deformable material that is capable of deforming under the force of normal human hand pressure, at least to an extent that allows the prosthetic foot to be inserted or removed from said foot shell. At the same time, the material should be elastic and rigid enough to snap back into its un-stretched configuration once the hand pressure has been removed. Further, the foot shell material should be rigid enough to adequately function as a foot shell when the foot shell (e.g. 320) is in turn used in a shoe, and/or when the user uses the foot shell without a shoe.

Implicit in the concept of a “slit” is of course, the fact that generally each slit will have two sides. Although some embodiments of the invention need not function with the aid of slit fastening devices such as (502) and (602), in other embodiments, the invention will use at least one slit fastening device such as (502) and/or (602). This slit fastening device will often be mounted on the foot shell, generally in a manner that straddles the one or more slits (e.g. 500, 600).

The slit fastening device will usually have an open and shut configuration. In the shut configuration, the slit fastening device will hold the two slides the slit or slits together, and in the open configuration, the slit fastening device will allow the sides of the slit or slits to separate.

The fastening device can thus comprise at least one latch or a clip, such as (602). In some embodiments, this at least one latch or clip can be removable from the foot shell. In other embodiments, the at least one latch or clip may be attached to at least one side of the slit on the exterior of the foot shell, such as via a hinge mechanism. In some embodiments, this at least one latch or clip may include a ring shaped latch or clip that fits around the upper rim (326) of the foot shell.

Although, in the embodiment shown in FIGS. 5 and 6, the at least one fastening device comprises a butterfly shaped latch or clip, where each wing of the butterfly shaped latch or clip is disposed on opposite sides of the slit, other latch or clip configurations may also be used. Indeed in some embodiments, the slit sides may even be tied up with laces, in which case eyelets may be provided on opposite sides of the foot shell. Zippers, Velcro, and other fastening material may also be used.

In a preferred embodiment, however, any slit closing mechanism should ideally be provided in a way that preserves the smooth exterior of the foot shell. This is helpful because the foot shell will normally be expected to fit into normal shoes, and unnatural protrusions can interfere with this process. Further it is often preferable to also have slits and closing mechanisms configured to provide a foot shell that preserves a natural flesh colored foot appearance.

As previously discussed, the dimensions (e.g. length of the one heel side slit(s) and/or the forefoot side slit(s)), and the deformability of at least the foot shell material proximate the slits, will optimally be selected so as to allow an average adult to rapidly remove the foot shell from the prosthetic foot, and to mount that foot shell or another foot shell configured according to the invention. Here “rapid” generally means a time span of about two minutes or less for the process, at least with some practice. The idea is to, at least for physically and mentally able prosthetic foot users, allow them to replace their foot shells quickly themselves. For less able prosthetic foot users, such as younger children or the elderly, the idea is to allow normal adult caregivers to perform this process.

Although even a single foot shell configured according to the invention will have utility—for example allowing the foot shell to be easily removed so that a fresh sock (514) can be placed on to the prosthetic foot, and then the foot shell then replaced, in a preferred embodiment, the invention will likely be most valued because it can be used to help the prosthetic foot user rapidly adapt to a variety of different walking conditions.

In particular, according to the invention, the foot shell may be used in a kit with one or more other foot shells also configured according to the invention. These different foot shells in this kit may, for example, comprise foot shells with varying heel and or sole heights (e.g. 424, 406) adapted to allow the prosthetic foot user to walk adequately barefoot, with low heel shoes, higher heel shoes, and the like.

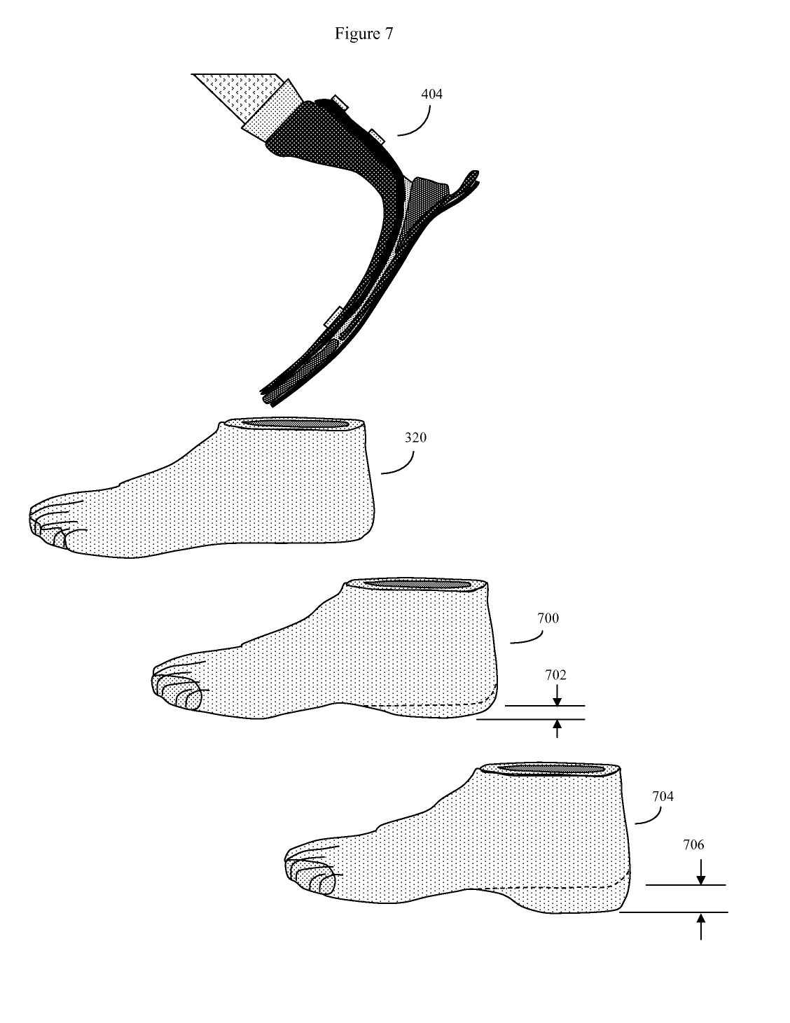

FIG. 7 shows a prosthetic foot (404) and a kit of three different foot shells, each with a somewhat different heel height, ranging from a low heel height (320), a medium heel height (700), (702), and a higher heel height (704), (706). The sole of the foot shell may also be adjusted in height as desired. Note that even the higher heel foot shell models such as (704) still retain a generally normal foot-like cosmetic appearance.

Thus by using a kit composed of at least two foot shells with at least different heel heights and optionally different sole heights, a prosthetic foot user can, for example, rapidly remove a first foot shell with a first heel height (such as 320), and rapidly mount a second foot shell with a second heel height (such as 704). Thus a user going out for the day, who later expects to enter a household where no shoes allowed, might start off the day by wearing foot shell (320). The user would carry foot shell (704) with them. They would then rapidly switch from foot shell (320) to foot shell (704) when entering the house where no shoes are allowed. The user can then switch back to using foot shell (320) when exiting the house.

Thus in such a kit configuration, it may often be desirable to provide at least one foot shell in the kit, such as (704), that has a heel height and/or sole height that is optimized for walking barefoot. At the same time, it may often be desirable to also provide at least one foot shell in the kit, such as (700) or (320) that has a heel height optimized for walking in shoes or footwear with a heel height of ¼″ or “⅜” or higher.

Returning to FIGS. 4 through 6, note that in some embodiments, the hollow interior of the foot shell (400) may additionally be configured to also accommodate the space taken up by a flexible fabric sock, such as (514) that covers at least the heel (420) and forefoot (422) portion of the prosthetic foot (404).

Further, the dimensions of the foot shells slit(s) (500) and/or forefoot side slit(s) (600), and the deformability of the foot shell material (412) (e.g. deformable material), may be further selected as to allow at least an average adult to remove the foot shell from the prosthetic foot, replace the sock (514) and then rapidly remount the foot shell, preferably within a time span of two minutes or less.

Note further that although, in a preferred embodiment, the invention's rapidly removable foot shell is configured to allow at least an average adult to remove the foot shell from the prosthetic foot, and to mount or remount the foot shell on the prosthetic foot, by hand and finger motions only without the use of external tools, in other embodiments various hand tools may be used for this process. If such hand tools are provided, it may be useful to provide the hand tools in a form that can easily be affixed to the foot shell or the prosthetic foot or leg, so that the user can always get rapid access to these tools.

In some embodiments, if the user additionally choses to use a thin flexible foot cover or “foot skin” on top of the foot shell, then the foot skin itself can be further used to help affix the foot shell to the prosthetic leg.

Other Advantages:

In addition to the uses and advantages previously discussed, the invention's rapidly replaceable foot shells have other advantages as well. Because the foot shell is designed for rapid release, the large amount of stress placed on a prior art foot shell during practitioner maintenance can be greatly reduced, thus prolonging the lifetime of the foot shell, at least in terms of numbers of removal and replacement cycles that the foot shell can withstand prior to the onset of foot shell deterioration. Thus for example, while a prior art foot shell might be expected to withstand only 1-5 cycles of practitioner removal and replacement, the rapidly replaceable foot shells of the present invention would withstand correspondingly more cycles of removal and replacement, potentially leading to longer foot shell effective life, at least in similar type use scenarios.

Further, because the foot shell user (or their caregiver) can themselves remove the invention's rapidly replaceable foot shell and inspect the interior surface of the foot shell for deterioration, the present invention allow allows for a higher degree of preventive maintenance than was previously possible using prior art foot shells.

Finally, although the foot shell will normally either be used for walking in more or less standard shoes, or walking barefoot, in some embodiments, the rapidly removable foot shell may be configured to resemble shoes, slippers, flip-flops, moccasins, sneakers, athletic shoes, or other type of footwear.

1. A rapid release hollow foot shell for a mechanical prosthetic foot, said foot shell comprising:
a hollow shell with an exterior approximately in the shape of a human foot, said hollow shell comprising a heel portion, a forefoot portion, and a sole portion;
said hollow shell having an interior configured to substantially surround at least the heel portion and the forefoot portion of a prosthetic foot comprising a heel portion, a forefoot portion, and a shank;
wherein said hollow shell has a heel height comprising the distance that said foot shell elevates the heel portion of said prosthetic foot when said foot shell is mounted on said prosthetic foot;
said hollow shell further comprising an upper rim disposed in a single closed loop configured to allow at least the shank portion of said prosthetic foot to extend outside said foot shell when said prosthetic foot is disposed inside said foot shell;
said foot shell additionally comprising at least one of:
1: at least one heel side slit of at least 1″ length extending from said upper rim towards the sole of said foot shell; or
2: at least one forefoot side slit of at least 1.5″ length extending from said upper rim towards the forefoot portion of said foot shell;
at least the upper portion of said foot shell proximate said heel side slit or said forefoot side slit further comprising a deformable material capable of deforming under the force of normal human hand pressure to an extent that allows said prosthetic foot to be inserted or removed from said foot shell;
wherein the dimensions of said at least one heel side slit or said at least one forefoot side slit, and the deformability of said deformable material, are further selected as to allow an average adult to remove said foot shell from said prosthetic foot, and to mount said foot shell on said prosthetic foot, within a time span of two minutes.

2. The foot shell of claim 1, wherein each slit has two sides, further comprising at least one slit fastening device mounted on said foot shell;
Said at least one slit fastening device having an open and shut configuration;
wherein said at least one slit fastening device is configured to hold said two slides of said at least one slit together when said at least one slit fastening device is in a shut configuration; and
wherein said at least one slit fastening device is configured to allow separation between said two slides of said at least one slit when said at least one slit fastening device is in an open configuration.

3. The foot shell of claim 2, wherein said fastening device comprises at least one latch or a clip, and wherein said at least one latch or clip are either removable from said foot shell, or is attached to at least one side of said slit on the exterior of said foot shell.

4. The foot shell of claim 3, wherein said at least one fastening device comprises a butterfly shaped latch or clip, each wing of said butterfly shaped latch or clip being disposed on opposite sides of said slit.

5. The foot shell of claim 1, used in a kit comprising a plurality of said foot shells with at least different heel heights, wherein a user of said kit can rapidly remove a first foot shell with a first heel height, and rapidly mount a second foot shell with a second heel height.

6. The foot shell of claim 5, wherein at least one foot shell in said kit has a heel height optimized for walking barefoot, and at least one foot shell in said kit has a heel height optimized for walking in shoes or footwear with a heel height of ¼″ or higher.

7. The foot shell of claim 1, wherein the interior of said foot shell is configured to additionally accommodate a flexible fabric sock covering at least the heel and forefoot portion of said prosthetic foot;
wherein the dimensions of said at least one heel side slit or said at least one forefoot side slit, and the deformability of said deformable material, are further selected as to allow an average adult to remove said foot shell from said prosthetic foot, to replace said sock, and to remount said foot shell on said prosthetic foot, within a time span of two minutes.

8. The foot shell of claim 1, wherein said foot shell is further configured to as to allow an average adult to remove said foot shell from said prosthetic foot, and to mount said foot shell on said prosthetic foot, by hand and finger motions only, and without the use of external tools.

9. A rapid release hollow foot shell for a mechanical prosthetic foot, said foot shell comprising:
a hollow shell with an exterior approximately in the shape of a human foot, said hollow shell comprising a heel portion, a forefoot portion, and a sole portion;
said hollow shell having an interior configured to substantially surround at least the heel portion and the forefoot portion of a prosthetic foot comprising a heel portion, a forefoot portion, and a shank;
wherein said hollow shell has a heel height comprising the distance that said foot shell elevates the heel portion of said prosthetic foot when said foot shell is mounted on said prosthetic foot;
said hollow shell further comprising an upper rim disposed in a single closed loop configured to allow at least the shank portion of said prosthetic foot to extend outside said foot shell when said prosthetic foot is disposed inside said foot shell;
said foot shell additionally comprising at least one of:
1: at least one heel side slit of at least 1″ length extending from said upper rim towards the sole of said foot shell; or
2: at least one forefoot side slit of at least 1.5″ length extending from said upper rim towards the forefoot portion of said foot shell;
at least the upper portion of said foot shell proximate said heel side slit or said forefoot side slit further comprising a deformable material capable of deforming under the force of normal human hand pressure to an extent that allows said prosthetic foot to be inserted or removed from said foot shell;
wherein each slit has two sides, further comprising at least one slit fastening device mounted on said foot shell;
said at least one slit fastening device having an open and shut configuration;
wherein said at least one slit fastening device is configured to hold said two slides of said at least one slit together when said at least one slit fastening device is in a shut configuration; and
wherein said at least one slit fastening device is configured to allow separation between said two slides of said at least one slit when said at least one slit fastening device is in an open configuration;
wherein the dimensions of said at least one heel side slit or said at least one forefoot side slit, and the deformability of said deformable material, are further selected as to allow an average adult to remove said foot shell from said prosthetic foot, and to mount said foot shell on said prosthetic foot, within a time span of two minutes;
said foot shell further being used in a kit comprising a plurality of said foot shells with at least different heel heights, wherein a user of said kit can rapidly remove a first foot shell with a first heel height, and rapidly mount a second foot shell with a second heel height.

10. The foot shell of claim 9, wherein said fastening device comprises at least one latch or a clip, and wherein said at least one latch or clip are either removable from said foot shell, or are attached to at least one side of said slit on the exterior of said foot shell.

11. The foot shell of claim 10, wherein said at least one fastening device comprises a butterfly shaped latch or clip, each wing of said butterfly shaped latch or clip being disposed on opposite sides of said slit.

12. The foot shell of claim 9, wherein at least one foot shell in said kit has a heel height optimized for walking barefoot, and at least one foot shell in said kit has a heel height optimized for walking in shoes or footwear with a heel height of ¼″ or higher.

13. The foot shell of claim 9, wherein the interior of said foot shell is configured to additionally accommodate a flexible fabric sock covering at least the heel and forefoot portion of said prosthetic foot;
wherein the dimensions of said at least one heel side slit or said at least one forefoot side slit, and the deformability of said deformable material, are further selected as to allow an average adult to remove said foot shell from said prosthetic foot, to replace said sock, and to remount said foot shell on said prosthetic foot, within a time span of two minutes.

14. The foot shell of claim 9, wherein said foot shell is further configured to as to allow an average adult to remove said foot shell from said prosthetic foot, and to mount said foot shell on said prosthetic foot, by hand and finger motions only, and without the use of external tools.

15. A method of rapidly reconfiguring the prosthetic foot and foot shell combination of a prosthetic foot user from a walking environment requiring a first combined prosthetic foot and foot shell heel height to a walking environment requiring a second combined prosthetic foot and foot shell heel height, said method comprising:
providing at least two foot shells, each foot shell comprising:
a rapid release hollow foot shell for a mechanical prosthetic foot, said foot shell comprising:
a hollow shell with an exterior approximately in the shape of a human foot, said hollow shell comprising a heel portion, a forefoot portion, and a sole portion;
said hollow shell having an interior configured to substantially surround at least the heel portion and the forefoot portion of a prosthetic foot comprising a heel portion, a forefoot portion, and a shank;
wherein said hollow shell has a heel height comprising the distance that said foot shell elevates the heel portion of said prosthetic foot when said foot shell is mounted on said prosthetic foot;
said hollow shell further comprising an upper rim disposed in a single closed loop configured to allow at least the shank portion of said prosthetic foot to extend outside said foot shell when said prosthetic foot is disposed inside said foot shell;
said foot shell additionally comprising at least one of:
1: at least one heel side slit of at least 1″ length extending from said upper rim towards the sole of said foot shell; or
2: at least one forefoot side slit of at least 1.5″ length extending from said upper rim towards the forefoot portion of said foot shell;
at least the upper portion of said foot shell proximate said heel side slit or said forefoot side slit further comprising a deformable material capable of deforming under the force of normal human hand pressure to an extent that allows said prosthetic foot to be inserted or removed from said foot shell;
wherein the dimensions of said at least one heel side slit or said at least one forefoot side slit, and the deformability of said deformable material, are further selected as to allow an average adult to remove said foot shell from said prosthetic foot, and to mount said foot shell on said prosthetic foot, within a time span of two minutes;
wherein a first foot shell from said least two foot shells is mounted on said user's prosthetic foot, and wherein said first foot shell is configured for a walking environment requiring a first combined prosthetic foot and foot shell heel height;
wherein a second foot shell from said at least two foot shells is not mounted on said user's prosthetic foot, and wherein said second foot shell is configured for a walking environment requiring a second combined prosthetic foot and foot shell heel height;
removing said first foot shell from said prosthetic foot, and mounting said second foot shell on said prosthetic foot;
thereby reconfiguring said prosthetic foot and foot shell combination of said prosthetic foot user to a walking environment requiring a second combined prosthetic foot and foot shell heel height.

16. The method of claim 15, wherein each slit has two sides, further comprising at least one slit fastening device mounted on said foot shell;
said at least one slit fastening device having an open and shut configuration;
wherein said at least one slit fastening device is configured to hold said two slides of said at least one slit together when said at least one slit fastening device is in a shut configuration; and
wherein said at least one slit fastening device is configured to allow separation between said two slides of said at least one slit when said at least one slit fastening device is in an open configuration;
wherein both said first foot shell and said second foot shells comprise said slit fastening devices;
further opening said first foot shell's slit fastening device before removing said first foot shell from said prosthetic foot; and
further opening said second foot shell's slit fastening device prior to mounting said second foot shell on said prosthetic foot, and closing said second foot shell's slit fastening device after mounting said second foot shell on said prosthetic foot.

17. The method claim 16, wherein said fastening device comprises at least one latch or a clip, and wherein said at least one latch or clip are either removable from said foot shell, or are attached to at least one side of said slit.

18. The method of claim 15, wherein the interior of said foot shell is configured to additionally accommodate a flexible fabric sock covering at least the heel and forefoot portion of said prosthetic foot;
wherein the dimensions of said at least one heel side slit or said at least one forefoot side slit, and the deformability of said deformable material, are further selected as to allow an average adult remove said foot shell from said prosthetic foot, to replace said sock, and to remount said foot shell on said prosthetic foot, within a time span of two minutes.

19. The method of claim 15, wherein said foot shell is further configured to as to allow an average adult to remove said foot shell from said prosthetic foot, and to mount said foot shell on said prosthetic foot, by hand and finger motions only, and without the use of external tools.

20. The method of claim 15, wherein said foot shell is further configured to resemble standard footwear.
